(19) United States
(12) Plant Patent Application Publication
(10) Pub. No.: US 2007/0044183 P1
(43) Pub. Date: Feb. 22, 2007

(54) Wheeling avocado

(76) Inventor: Neal Palmer Brooks, Homestead, FL (US)

Correspondence Address: MICHAEL C. CESARANO, SUNTRUST INTERNATIONAL CENTER, 28TH FLOOR, 1 S.E. 3RD AVENUE, MIAMI, FL, 33131-1714, US

(21) Appl. No.: 11/205,815

(22) Filed: Aug. 17, 2005

(51) Int. Cl. A01H 5/00

(52) U.S. Cl. PLT/200

(57) ABSTRACT

The Wheeling avocado, which is a result of cross-pollination of the Monroe and the Brooks Late varieties, is of medium size, averaging 12-16 ounces and being about 4¾ inches in length, and has overall good eating qualities. The fruit matures and can be picked in late February and March. The fruit is an ovate berry that has a hard shelled, smooth textured, exocarp that is dark green.

### BACKGROUND OF THE INVENTION

During the decades of the '70's, '80's, and extending into the '90's, attempts were made to grow avocado plants in an area of Southwest Florida near the town of Immokalee. Cold winter weather and repeated freezes eventually overcame most of the attempts by killing the trees or their fruit before they could reach maturity. Two varieties, however, were able to reach maturity. These were the Monroe, a type B flower, and the Brooks Late, a type A flower.

The Monroe was larger, and could be used by customers who want large slices. The Brooks Late was the latest maturing, but was too small for commercial sales. Cross pollination of these two varieties was attempted in an effort to produce an avocado having the best characteristics of each.

Several hundred cross-pollinated fruits were harvested and the seeds were planted. From those seeds, plants that did not show healthy growth, or those which showed signs of being overly susceptible to the scab fungus disease were discarded. Of the remaining plants, the “Wheeling” variety matures in February and early March, and shows promise for commercial development.

### BRIEF SUMMARY OF THE INVENTION

All color descriptions employ the Munsell Color Charts for Plant Tissues, GretagMacbeth LLC, 617 Little Britain Road, New Windsor, N.Y. 12553-6148. The complete Munsell notation for any chromatic color is written: Hue (Value/Chroma).

The name of this new and distinct variety of avocado plant is ‘Wheeling’ Avocado tree. The botanical name of the observed plant is Persea americana Miller.

The tree is now two years old. It differs from similar varieties of avocado because of the lateness of the maturity of the fruit. The new avocado variety seems to express more resistance than other late varieties do to Cercospora purpurea and Colletotrichum gloeosporoides pathogens. This variety's tolerance to frost is believed to be strong, as would be indicated by its genetic heritage.

### BRIEF DESCRIPTION OF THE DRAWINGS

FIG. 1 shows leaf detail and a portion of the trunk at a lower portion of the avocado tree.

FIG. 2 shows the color and internal configuration of the fruit and the seed.

FIG. 3 shows the middle and upper portion of the avocado tree.

FIG. 4 shows an alternative view of the middle and upper portions of the avocado tree.

FIG. 5 shows the scale in inches of a typical fruit of the tree measured against a ruler.

FIG. 6 shows the sparse foliage near the upper portion of the avocado tree.

FIG. 7 shows the color and internal configuration of the fruit of FIG. 2 under marginally different lighting conditions.

FIG. 8 shows the dense foliage at the lower portion of the avocado tree.

### DETAILED BOTANICAL DESCRIPTION

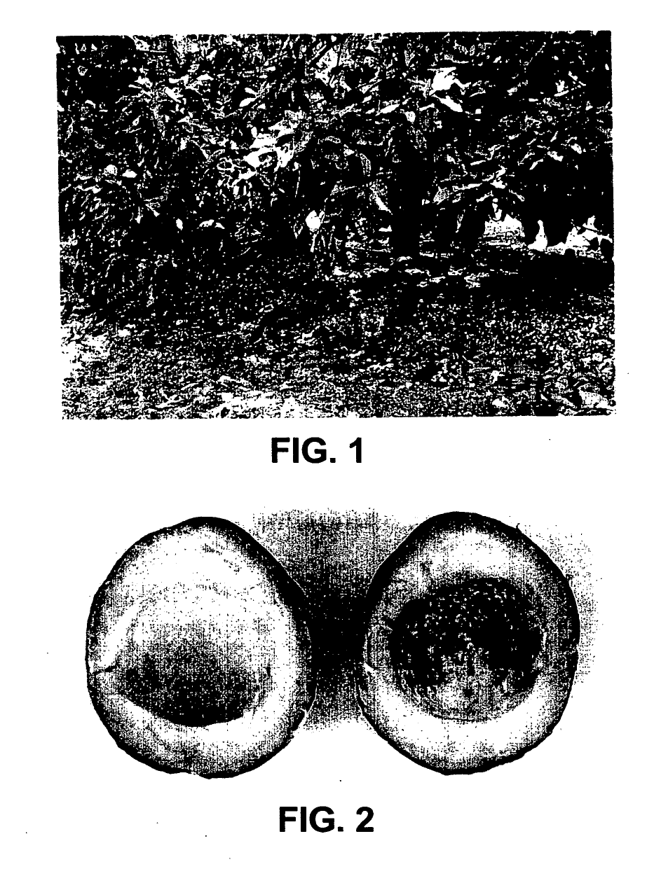

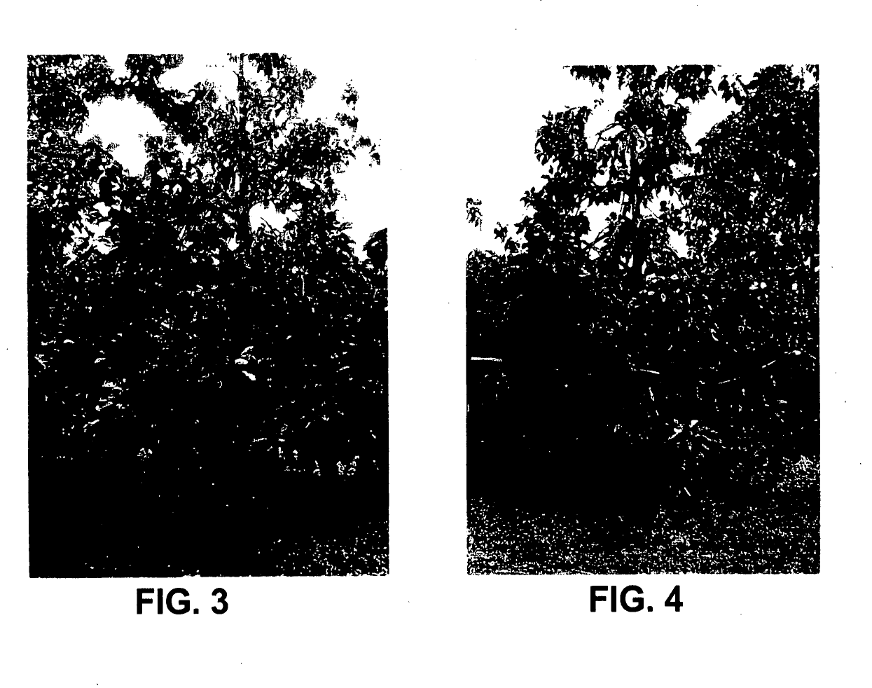

The tree is a vigorous upright growing tree, 20 feet high with a spread of 13 feet, as is shown in FIGS. 1, 3, 4, 6 and 8. As shown in FIG. 1, the diameter of the main trunk is 6-8 inches, 16 inches above the ground, tapering as it extends straight and upward to the pinnacle of the tree. Pendant branches of 2-3 inches in diameter split off of the vertical trunk, these in turn giving way to smaller and smaller branches growing in pendant fashion. One year of wood averages one inch in diameter.

The bark on the trunk and branches is light in color 2.5 Y (6/2). One year old wood is somewhat darker at 7.5 YR (5/2). New shoots average ½ inches in diameter and are greenish yellow 2.5GY (8/6).

The foliage is of heavy density near the ground, becoming sparse toward the upper regions of the tree. Leaves are large and dark green, as appears in FIGS. 1, 3, 4, 6 and 8. The leaves are alternate, entire and pinnately veined. They are elliptic, narrowly acute at the apex and acute at the base. The leaves are 4 to 16 inches in length, pubescent when young, becoming smooth and leathery when mature. The upper surface of the leaf exhibits a range of dark green from 7.5 GY (4/3 to 4/4). The underside of the leaf ranges from 5 GY (5/4 to 6/4). Leaf veins are a greenish yellow color of 2.5 GY (7/8), and the petioles are 2.5 GY (7/6). The leaves are not fragrant when crushed.

Multiple flowers on axillary panicles are borne in a pseudoterminal position and the central axis of the inflorescence terminates in a shoot. Flowers are perfect, with 12 stamens, 9 of which are functional, each having 4 pollen chambers. The single pistil has one carpel with one ovule. The flowers are ⅜ to ½ inch in diameter. They lack petals but have 2 whorls of 3 perianth lobes, and are pubescent. The color of the lobes and the pistil is light green 2.5 GY (8/4), the sepals are darker at 2.5 GY (7/6), and the anthers are yellow 2.5 Y (8/10). The flowers are not fragrant. The tree blooms in April and the blooms are of the B type. Fruit matures and can be picked in March of the following year.

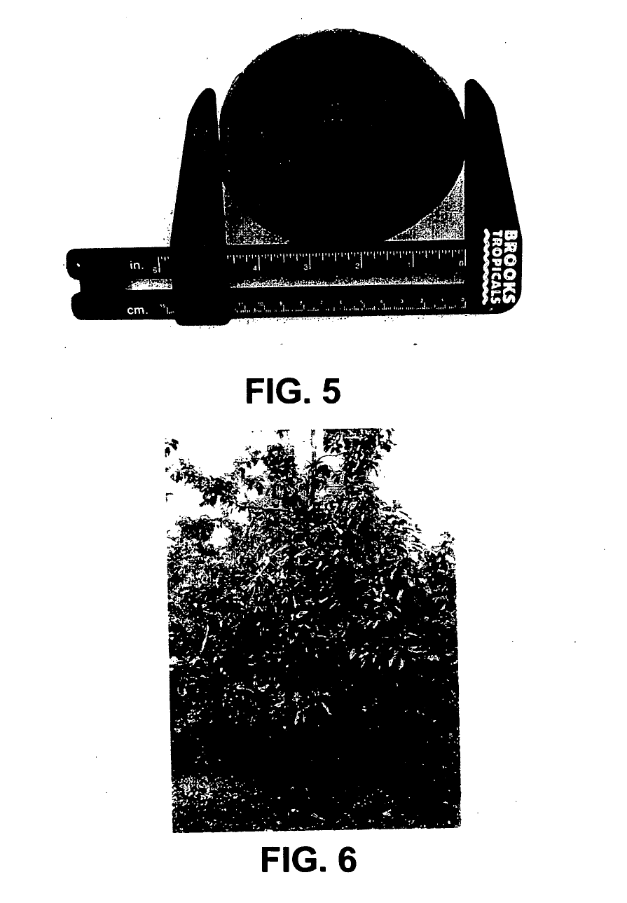

FIG. 5 shows the outside of the fruit, which is an ovate berry that matures in late February and March. It has a thick ( 1/16 inch) hard shelled exocarp, which is smooth textured, and dark green 5GY (4/6). The fruit weighs 12-16 ounces and averages 4¾ inches in length, as shown in FIG. 5.

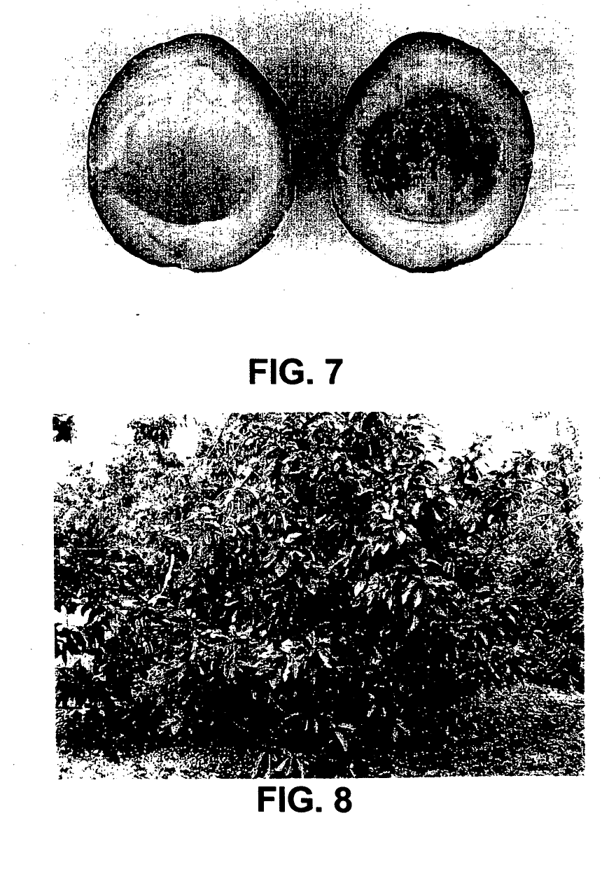

In FIGS. 2 and 7, the interior of the fruit is seen to have a mesocarp color that is green near the shell 5GY (7/8) and yellow near the seed cavity 5Y (8/10). Lenticels are numerous (100-200 per square inch) on the fruit and are light yellowish green in color 2.5 GY (7/10). The fruit also has an excellent flavor.

The seed is tight in the cavity, oblate, and is 1¾ to 2 inches long and is covered by a dark brown endocarp 5 YR (4/4). The Cotyledons are textured and light brown in color 5 YR (6/8).

We claim:

1. A new and distinct variety of avocado tree substantially as described and illustrated and characterized as to novelty by its overall good eating qualities, its medium size and its being ready to be picked in late February and March.
